(19) United States
(12) Patent Application Publication
(10) Pub. No.: US 2020/0360035 A1
(43) Pub. Date: Nov. 19, 2020

(54) Device and Methods For Laser Tongue Debridement For Oral Malodor

(71) Applicant: Valam Corporation, New York, NY (US)

(72) Inventors: Yosef Krespi, New York, NY (US); Victor Kizhner, New York, NY (US); Karen Wilson, New York, NY (US)

(21) Appl. No.: 16/877,195

(22) Filed: May 18, 2020

Related U.S. Application Data
(60) Provisional application No. 62/849,152, filed on May 17, 2019.

(51) Int. Cl. A61B 17/24; A61N 5/06

(52) U.S. Cl. CPC A61B 17/244; A61N 2005/067; A61N 2005/0606; A61N 5/0624

(57) ABSTRACT

There is provided a method of biofilm reduction from the tongue which includes the steps of applying water to a selected area of biofilm and applying an Er, CR: YSGG solid state laser to the selected biofilm area from a hand held device. The method includes generating cavitation to the selected area of biofilm by the application of water and the application of the Er, CR: YSGG solid state laser to result in biofilm disruption without tissue injury. The biofilm disruption produces a reduction of the biofilm over a period of time, with a reduction in aerobic and anaerobic bacteria. A comparison of digital image analyses of tongue coatings over a period of time can determine the reduction in biofilm.

### CROSS REFERENCE TO RELATED APPLICATIONS

This application claims the benefit of and priority to U.S. Provisional Patent Application No. 62/849,152 filed on May 17, 2019 and incorporated herein by reference in its entirety.

### BACKGROUND

Oral malodor is a poorly understood phenomenon; its diagnosis, treatment, and follow-up is elusive. This leads to a reduction in social activity and an increased social burden for patients, which often leads to frustration. Without clear diagnostic and treatment plans, patients typically resort to short-lasting measures that generally merely mask the malodor. Oral malodor is the most common form of malodor—the teeth, gums, tongue, and tonsils act as the main culprits. The tongue has been shown to harbor bacteria similar to that of dental biofilm; therefore, similar methods can be applied to treat both types of bacteria. There is a need for a method of removing biofilm which causes malodor and a device used in practicing the method of biofilm removal.

### SUMMARY

The present invention is a method of biofilm reduction from the tongue of a patient having halitosis. The method includes applying water to a selected area of biofilm and applying an Er, CR: YSGG solid state laser to the selected area of biofilm. The method of the present invention includes generating cavitation to the selected area of biofilm by the application of water and the application of the Er, CR: YSGG solid state laser to result in biofilm disruption without tissue injury. The biofilm disruption produces a reduction of the biofilm over a period of time. In the method of the present invention, the solid state laser is applied at 4 watts and at a 20 Hz frequency for 5 minutes. After thirty days, the patient has a follow up appointment to review the treatment progress.

With the method of the present invention, there is aerobic and anaerobic bacterial reduction to the selected area. In the method, the reduction of the biofilm is determined by comparison of digital image analyses of tongue coatings over a given period of time. Changes in color of the digital images can be measured, including the types of color and percentages of each, from foul yellow/brown colors to healthy pink or salmon colors.

In the method, the solid state laser is applied from the tip of a hand held device connected to a power source.

### BRIEF DESCRIPTION OF THE DRAWINGS

Some embodiments of the invention, and of making and using the invention, as well as the best mode contemplated of carrying out the invention, are described in detail below, by way of example, with reference to the accompanying drawings, in which like reference characters designate like elements throughout the several views, and in which:

FIG. 1 is an illustration of Halimeter readings with mean before and after treatment with one month follow up.

FIG. 2 is an illustration of the aerobic bacterial reduction, before and after treatment;

FIG. 3 is an illustration of the anaerobic bacterial reduction, before and after treatment;

FIG. 4 is a chart of the patient reported VAS Pain score of laser treatment;

FIG. 5 is a chart of organoleptic judges score with mean before and after treatment;

FIG. 6 is a chart of HALT scores pre-treatment with one month follow up;

FIG. 7 is an illustration of the process for laser tongue debridement in office treatment;

FIG. 8 is an illustration of a tongue map with particular treatment area, typical for halitosis;

FIG. 9 is a chart of tongue color analysis over time;

FIGS. 10A and 10B illustrates laser tongue debridement, before and after treatment, with a gain in healthy colors and a reduction in foul for a patient;

FIGS. 10C and 10D illustrate a second set of laser tongue debridement, before and after treatment, with a gain in healthy colors and a reduction in foul for a second patient;

FIG. 11 is an image of the device with special lens tip of the present invention for performing laser tongue debridement.

FIG. 12 is an illustration of the color breakdown of the tongue using the imaging application on a phone or handheld device.

FIG. 13 is a second illustration of the color breakdown of the tongue using the imaging application on a phone or handheld device.

### DETAILED DESCRIPTION

The following more detailed description of the invention is intended to be read in the light of, and in context with, the preceding summary and background descriptions but without being limited by the preceding descriptions.

As stated above, the tongue has been shown to harbor bacteria similar to that of dental biofilm; therefore, similar methods can be applied to treat both types of bacteria. As this biofilm spreads with the reduction of inflammatory changes, it is expected that the tongue color will change alongside subjective reductions in bacterial activity. Bacterial activity can be measured through using direct cultures with CFUs, and the bacterial breakdown of amino acids resulting in production of volatile sulfur compounds (VSCs) can be measured by a halimeter. The HALT questionnaire used in this study is designed to measure subjective patient well-being and is correlated to objective measures. Further, the Er,Cr:YSGG solid-state laser has been shown to be effective in promoting biofilm reduction. As the laser is absorbed by water, the dual action of the water and the laser will generate an optical breakdown (cavitation) resulting in biofilm disruption without tissue injury. The Er,Cr:YSGG laser has been shown to be more effective than other lasers, such as the Nd:YAG laser, particularly against the Enterococcus faecalis and Candida albicans biofilms.

The methods used in the study with the present invention were as follows: A randomized controlled prospective study with IRB approval was performed. The enrollment criteria included adults with oral malodor scoring>2 on an organoleptic test and without any signs of gingival inflammation or systemic halitosis. In total, 54 patients met the enrollment criteria; 30 were placed in the laser tongue debridement (LTD) group, and 24 were placed in the control group. Patients in the LTD group were treated with the Er,Cr:YSGG laser (Waterlase Express, Biolase Irvine, Calif.) at 4W 20 Hz for 5 minutes, and members of the control group received a mechanical debridement using a soft brush on their first visit. A one-month follow-up appointment for data collection was scheduled for both groups. The following data was collected: concentrations of viable aerobic and anaerobic bacteria from tongue dorsum, results of organoleptic tests according to the Rosenberg protocol, VSC halimeter readings, responses to the HALT patient questionnaire measuring the treatments' quality-of-life impacts, reported patient tolerances for the laser treatment (using a VAS pain scale), and image analyses of tongue coatings (taken with single device under the same lighting conditions and analyzed using similar methods). As halimeters measure only the VSCs produced mainly by the Porphyromonas, Prevotella, Actinobacillus, and Fusobacterium species, the cultures were divided into aerobic and anaerobic groups. Digital tongue images obtained were divided into “healthy” and “foul” color groups.

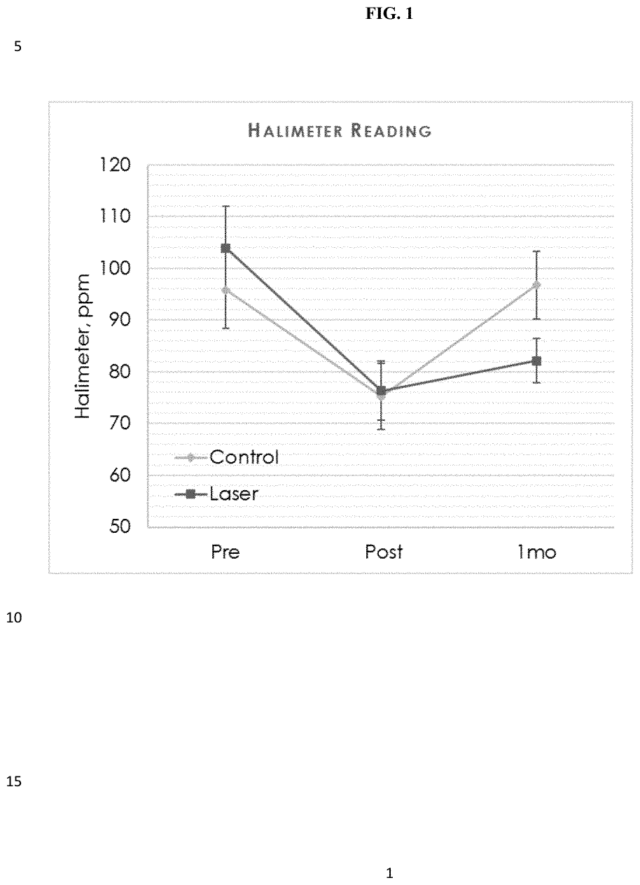

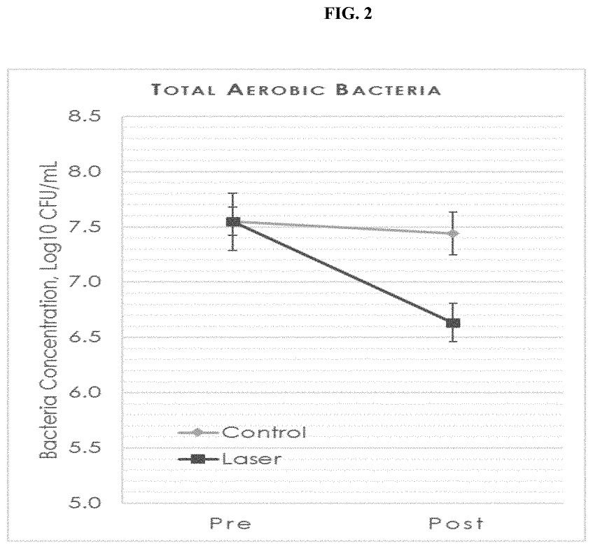

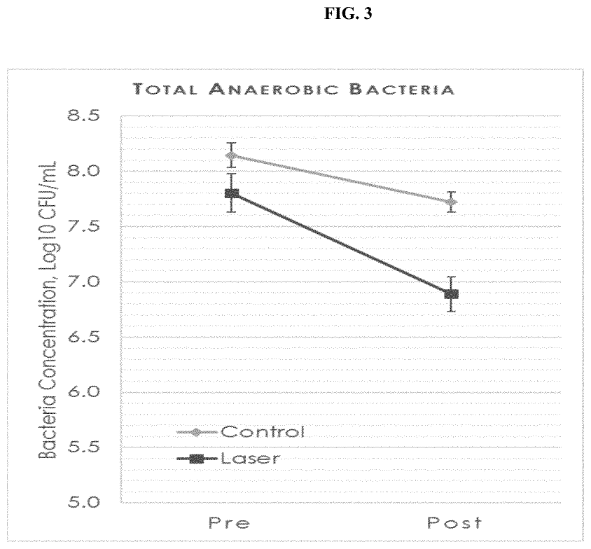

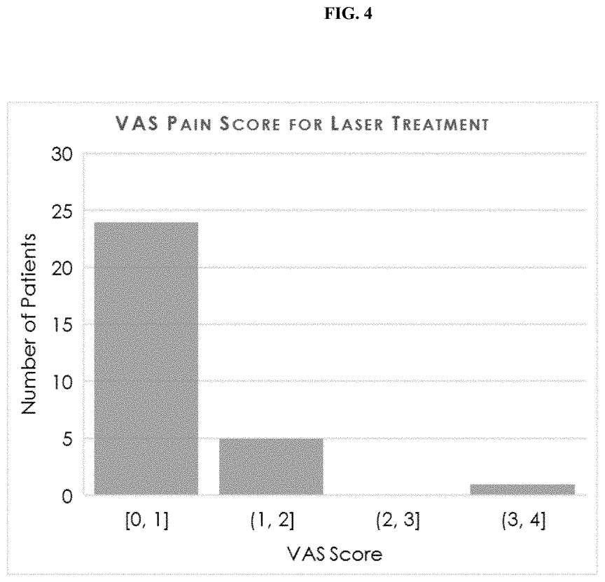

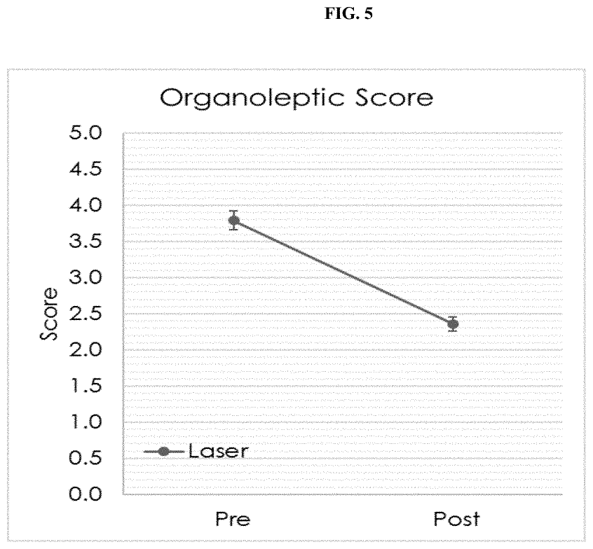

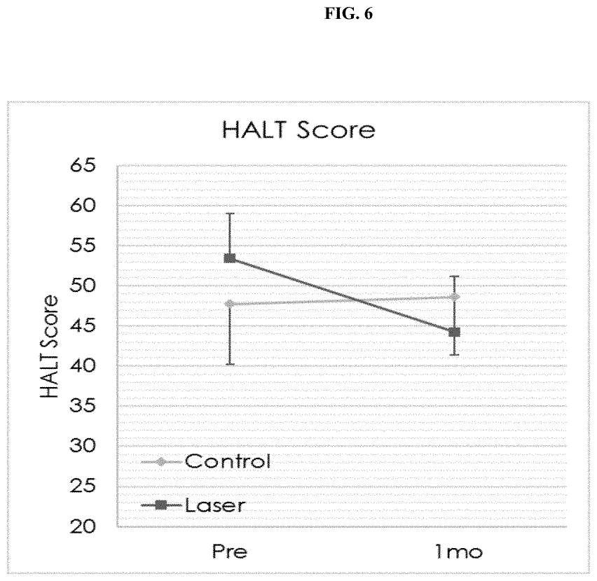

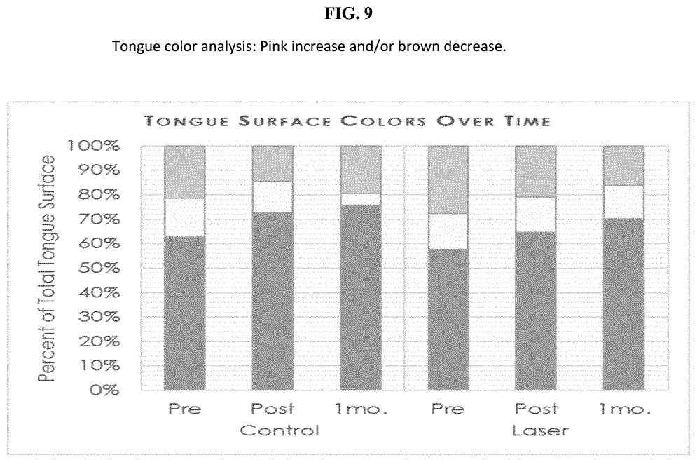

The results from the above method were as follows: In all, 35 patients completed the study upon their follow-up appointments; most participant dropouts were from the control group. Halimeter scores also reduced immediately following treatment in both groups (FIG. 1). LTD produced a more significant response that was still present at the follow-up, but this result was not found in the control group (t-test with P<0.03 for all comparisons). LTD caused a significant log reduction of ˜1 (t-test P<0.01) in participant bacterial count (FIGS. 2 and 3). The VAS scores were negligible (FIG. 4); a single patient reported discomfort due to TMJ muscle spasm. The organoleptic score of the laser group decreased from 3.75 to 2.3 (P<0.05) (FIG. 5). HALT scores decreased from 53 to 45 (P=0.01) (FIG. 6). Color analysis showed an increase in healthy pink colors (t-test P=0.02 for pink salmon) and a decrease in foul brown colors in the laser therapy group (FIG. 9). Interestingly, foul colors were more often detected with a high halimeter and organoleptic readings which remained present after one month. Halitosis is shown to be reducible through several methods. LTD results are sustained for at least one month, and it is easily tolerated. Tongue color analysis proves to be objective and simple to perform, allowing for treatment monitoring.

It is concluded that LTD offers reduction in oral malodor burden. This reduction is demonstrated by series of objective and subjective testings and improves patients quality of life. The multifactorial nature of halitosis is shown to be improved by several methods. LTD is sustained at least for 1 month and is easily tolerated. Tongue digital color analysis proves to be an objective, simple method to perform analysis, allowing for monitoring of treatment

Referring to FIG. 1, there is an illustration of Halimeter readings with mean before and after treatment with one month follow up. The Halimeter readings of the control group revert back to pre-treatment levels one month after the control treatment. However, the Halimeter readings of those receiving the laser treatment show a significant reduction is still maintained one month after treatment.

With FIG. 2 is an illustration of the aerobic bacterial reduction, before and after treatment and FIG. 3 is an illustration of the anaerobic bacterial reduction, before and after treatment. The charts show the reduction in aerobic bacteria and anaerobic bacteria as a result of the laser treatment.

FIG. 4 is a chart of the patient reported VAS Pain score of laser treatment, indicating very low pain scores for the patients. With reference to FIG. 5, there is the chart of organoleptic judges score with mean before and after treatment, indicating a decrease in the organoleptic judges score for the laser debridement treatment post treatment.

FIG. 6 is a chart of HALT scores pre-treatment and with one month follow up. The HALT scores decreased for the treatment group compared with the control group.

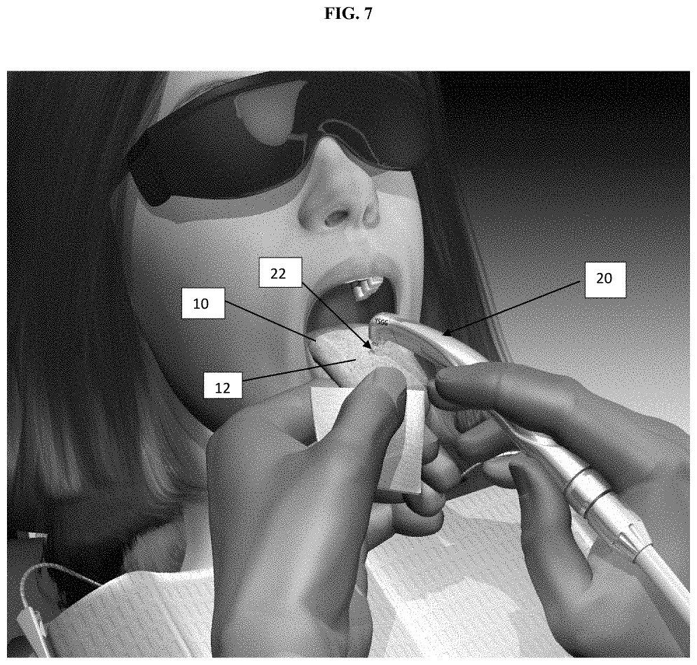

FIG. 7 illustrates the process for laser tongue debridement of the present invention in office treatment. As shown in the illustration, a patient with halitosis or malodor and a biofilm 12 on her tongue 10 is ready for treatment by a medical professional. The medical professional uses a hand held Er,Cr:YSGG laser device 20 with the specialized tip 22 to deliver laser energy to the selected area that has biofilm 12.

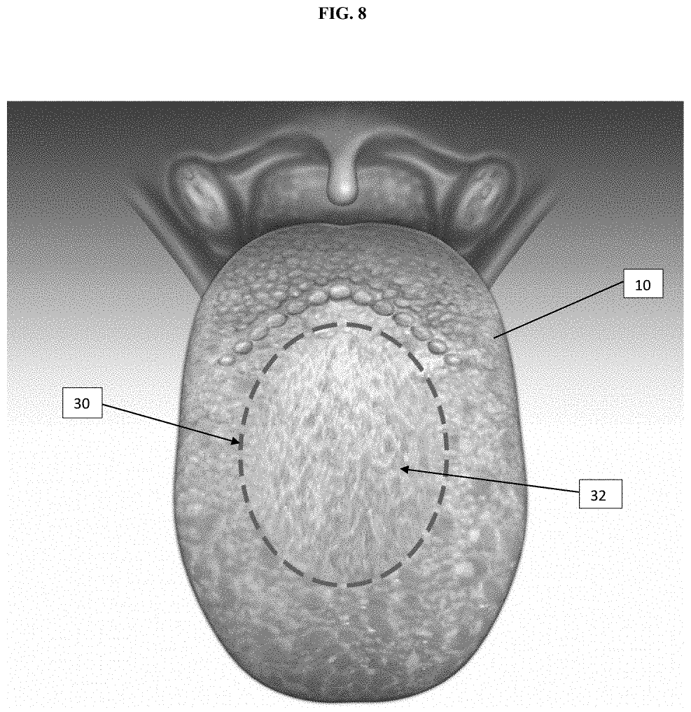

Referring now to FIG. 8, there is an illustration of a tongue 10, mapped with a particular treatment area 32 that is typical for halitosis as indicated by the dashed circle area 30. It is within this circled area 30 that the medical professional selects for treatment of the patient with the process of the present invention, although other areas are within the scope of the invention as may be needed by a particular patient. As shown previously with FIG. 7, the medical professional places the tip 22 of the hand held device 20 over and within this area 30 to deliver the laser treatment.

FIG. 9 is a chart of tongue color analysis over time. The chart depicts the percentage of the total tongue surface that is covered by a favorable color of pink and that of undesirable color brown/yellow, indicating biofilm causing malodor. The left three vertical bars represent the control group and the right three bars represent the group of patients treated with the laser debridement process of the present invention. The three vertical bars are shown for pre-treatment, post treatment and one month after treatment. As indicated in the chart, the patients that received the laser treatment process had an increase in favorable pink color.

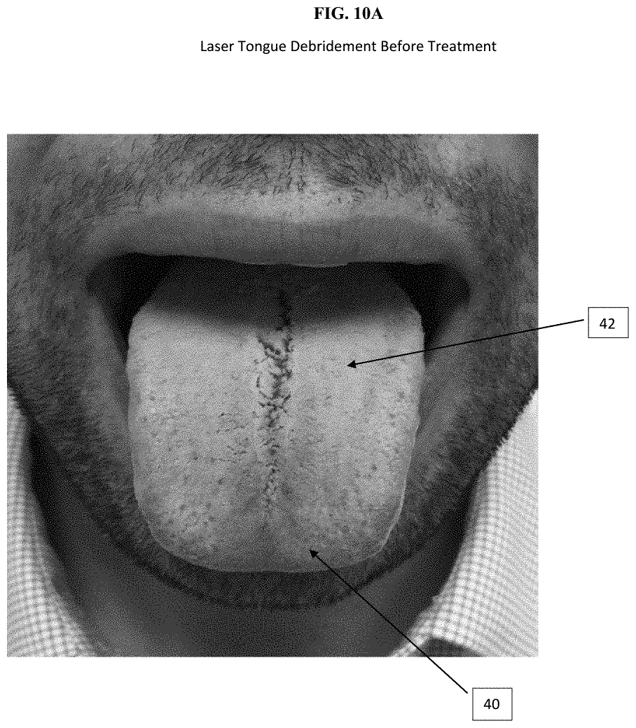

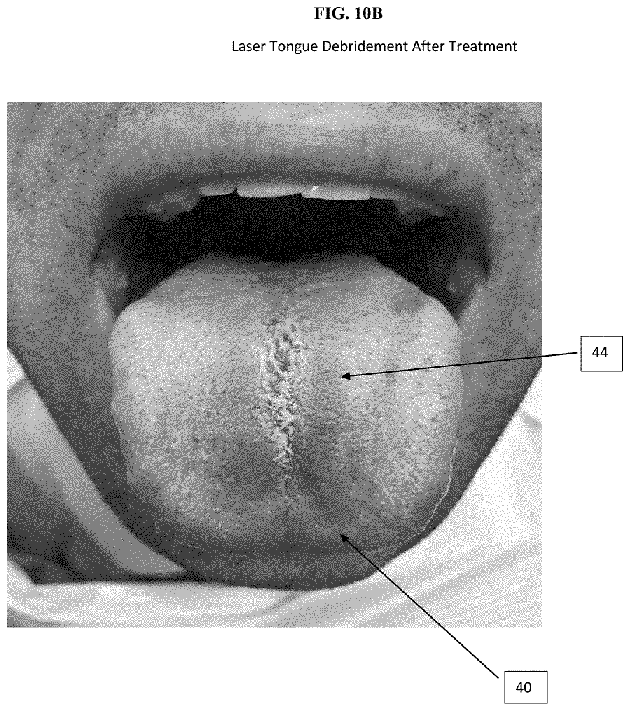

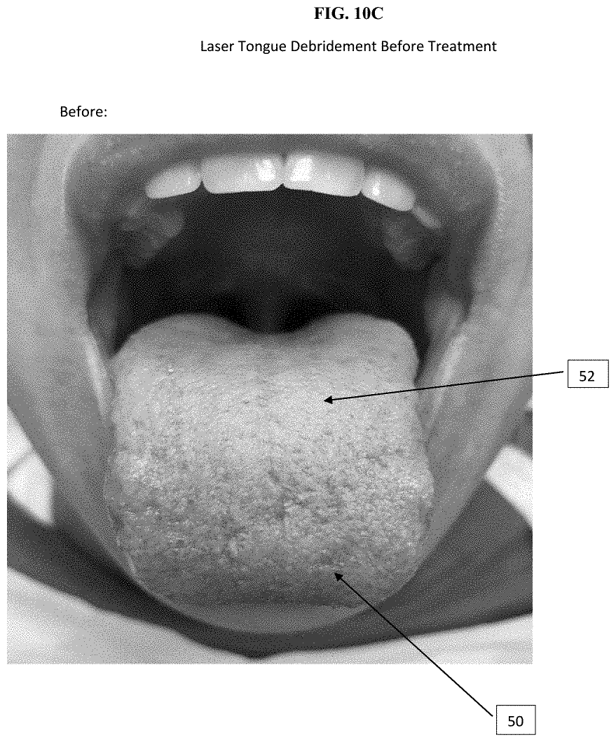

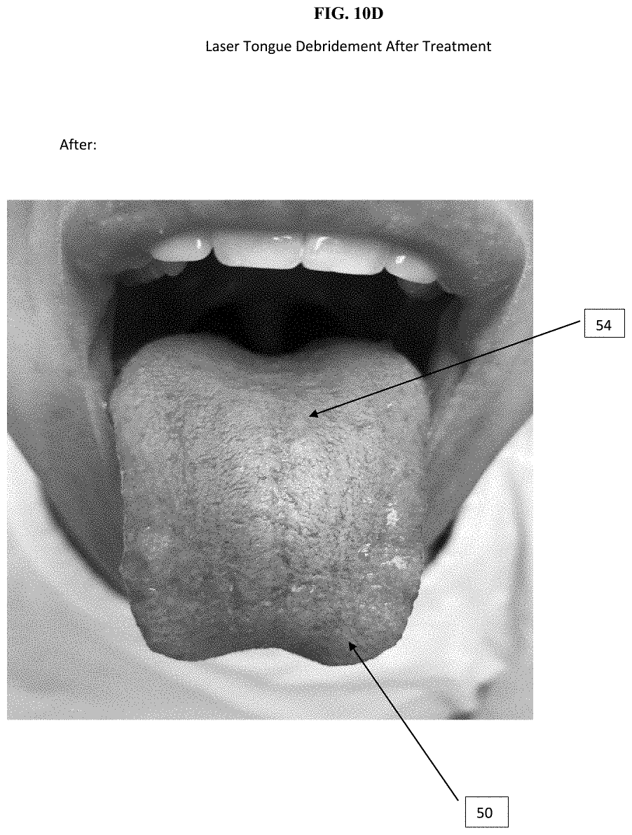

As show with reference to FIGS. 10A and 10B, there is a noticeable and visible result with the laser tongue debridement process of the present invention. FIG. 10A shows before and FIG. 10B shows after images of a patient who has undergone the laser tongue debridement method of the present invention. In the before photos, the patient's tongue 40 is coated with biofilm 42 with colors in the shades of brown and yellow. The patient is then treated with the method according to this invention. In the after image, the patient's tongue 40 is shown with significant increase in healthy colors 44 and a reduction of the foul, brown or yellow colors. Likewise, FIGS. 10C and 10D illustrate a second set of laser tongue debridement treatment, with images of the patient before the treatment (FIG. 10C) and after the treatment (FIG. 10D), with the process of the present invention. The before image indicates biofilm 52 on the patient's tongue 50 with a presence of yellowish colors and as shown again in the after image, the patient's tongue 50 has a gain in healthy colors 54 and a reduction in foul.

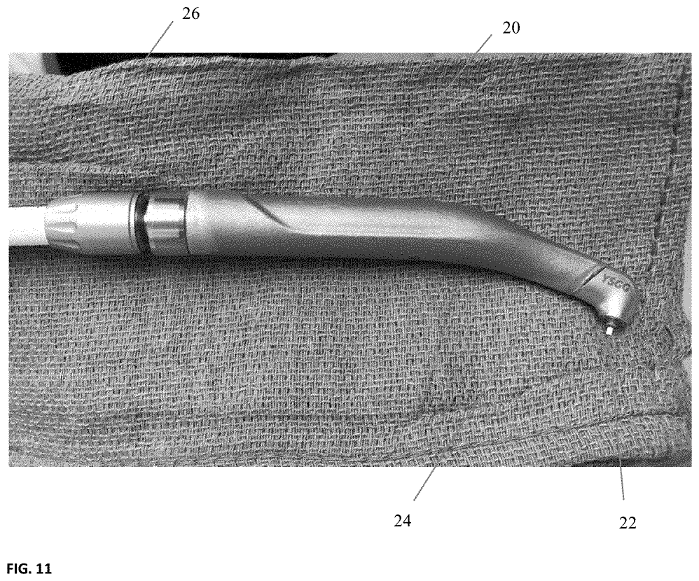

FIG. 11 is an image of the device 20 with special lens tip 22 of the present invention for performing laser tongue debridement. The hand held device 20 has an angled head section 26 culminating in the tip 22 for delivering laser energy to the tissue surface to be treated. The device 20 is connected via input line 26 to a power supply.

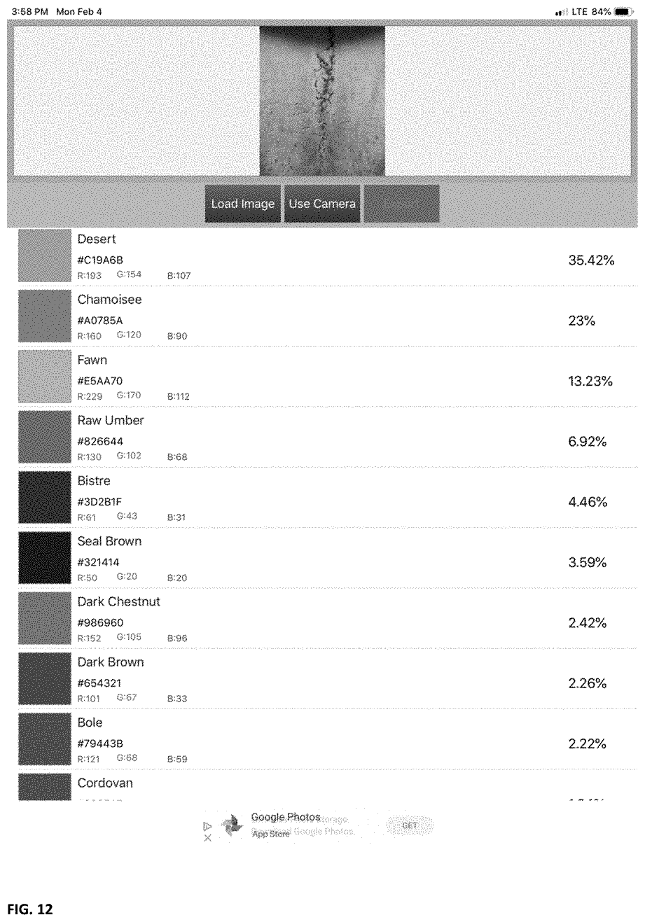

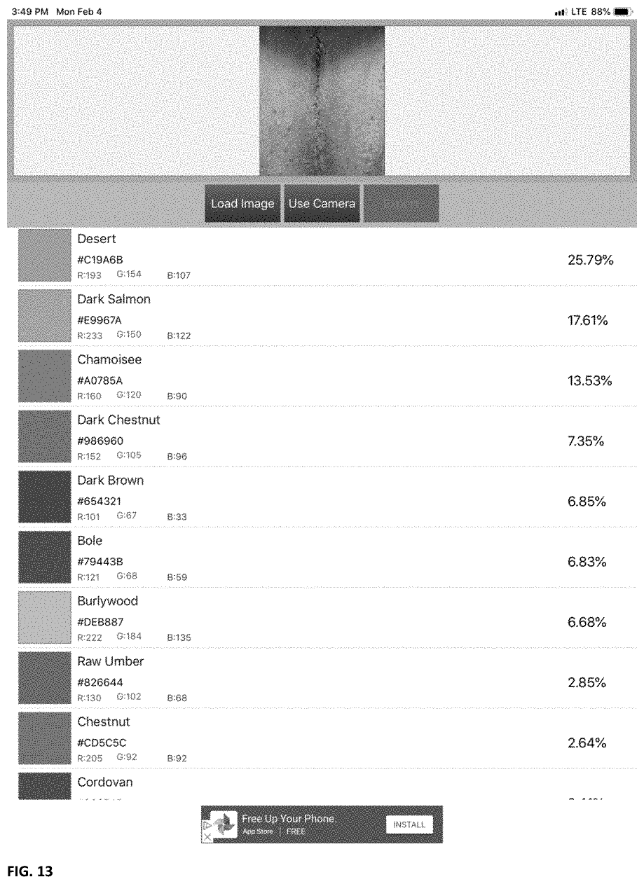

FIG. 12 is an illustration of the color breakdown of the tongue using the imaging application on a phone or handheld device, or other camera application which produces digital images. This is taken before treatment with the present invention, with a high percentage of the brown/yellow desert color present on the tongue. With reference now to FIG. 13, there is shown a second illustration of the color breakdown of the tongue using the imaging application on a phone or handheld device. The illustrations are shown as taken after treatment with the present invention, indicating a reduction in the percentage of the brownish desert color present on the tongue. There is also an increased percentage of the salmon color, indicating more healthy areas with reduced biofilm.

While illustrative embodiments of the invention have been described above, it is, of course, understood that many and various modifications will be apparent to those of ordinary skill in the relevant art, or may become apparent as the art develops. Such modifications are contemplated as being within the spirit and scope of the invention or inventions disclosed in this specification.

What is claimed is:

1. A method of biofilm reduction from the tongue,
applying water to a selected area of biofilm;
applying a Er, CR: YSGG solid state laser to said selected area of biofilm;
generating cavitation to said selected area of biofilm by said application of water and said application of said Er, CR: YSGG solid state laser to result in biofilm disruption without tissue injury; said biofilm disruption producing a reduction of said biofilm over a period of time.

2. The method according to claim 1, wherein said solid state laser is applied at 4 watts.

3. The method according to claim 1, wherein said solid state laser is applied at 20 Hz frequency.

4. The method according to claim 1, wherein said solid state laser is applied for 5 minutes.

5. The method according to claim 1, wherein there is aerobic bacterial reduction to said selected area.

6. The method according to claim 1, wherein there is anaerobic bacterial reduction to said selected area.

7. The method according to claim 1, wherein said reduction of said biofilm is determined by comparison of digital image analyses of tongue coatings over said period of time.

8. The method according to claim 1, wherein said solid state laser is applied from the tip of a hand held device.

9. The method according to claim 1, wherein said period of time is thirty days.
